(19) United States
(12) Patent Application Publication
(10) Pub. No.: US 2005/0080490 A1
(43) Pub. Date: Apr. 14, 2005

(54) Acetabular liners for revision hip surgery

(76) Inventor: Morton Bertram III, Naples, FL (US)

Correspondence Address: John G. Posa;Gifford, Krass, Groh, Sprinkle,, Anderson & Citkowski, P.C., 280 N. Old Woodward Ave., Suite 400, Birmingham, MI, 48009-5394, US

(21) Appl. No.: 10/961,805

(22) Filed: Oct. 8, 2004

Related U.S. Application Data
(60) Provisional application No. 60/509,728, filed on Oct. 8, 2003.

(51) Int. Cl. A61F 2/34

(52) U.S. Cl. 623/22.28

(57) ABSTRACT

Liner, preferably made of polyethylene, specifically designed for re-cementation into an existing ace tabular shell. Whereas existing polyethylene liners have not been designed to be re-cemented, this invention includes various features, particularly on the posterior aspect of the liner, to resist pull-out, minimize rotational torsion, and to ensure proper cement mantle thickness. In the preferred embodiment, the polyethylene liner is disposed within a shell such that the cement locks into features provided on the posterior aspect of the liner which remains in contact with cement. Liners according to the invention further preferably include an extended rim to improve cement pressurization, as well as features to resist torsion as well as pullout, and spacers to ensure proper cement mantle thickness

This application claims priority from U.S. Provisional Patent Application Ser. No. 60/509,728, filed Oct. 8, 2001, the entire content of which is incorporated herein by reference.

### FIELD OF THE INVENTION

This invention relates generally to joint replacement and, in particular, to ace tabular liners for revision hip surgery.

### BACKGROUND OF THE INVENTION

Revision hip surgery for a loosened, cemented cup is technically straightforward, including revision surgery. However, because cemented cups fail at an unacceptably high rate, reconstructive hip surgeons in this country, and the companies that develop related products, have shifted their efforts to uncemented cups and modular liners. Although the longevity and life expectancy of the porous ingrowth shell has been excellent, surgeons and patients are still faced with the problem of polyethylene wear and polyethylene failure.

A dilemma arises when faced with a well-fixed porous ingrowth ace tabular shell and a failed modular polyethylene liner. In many instances, these shells have osteolysis surrounding them and it is a very difficult reconstructive procedure to revise a well-ingrown porous ingrowth acetabular shell. For this reason, it has become popular to leave the shell in place and cement in a new liner to the well-fixed ingrowth acetabular shell

Unfortunately, surgeons typically do not have polyethylene liners in inventory that will fit the shell perfectly. Most of the implants are outdated or the lines have been discontinued. Therefore, it is difficult if not impossible to find a matching polyethylene liner for a given well-fixed ingrowth shell.

For this reason, some surgeons have elected to cement the polyethylene liner into an existing shell. To do this, the surgeon has to use a high-speed burr and roughen up the surface of the polyethylene liner so that it may be cemented into the metal shell. The surface roughening enhances the fixation of the cement bond.

Studies have been done to show that the pullout strength of the liners is excellent and, in fact, if the liner is placed properly the use of cement is comparable to standard locking mechanisms. Journal articles further indicate that if properly designed and modified, these liners have superior pullout strength even compared to existing modular liners. Liners without modification, however, have been shown to pull out of the metal shell into which they have been re-cemented.

### SUMMARY OF THE INVENTION

This invention broadly resides in a liner, preferably made of polyethylene, specifically designed for re-cementation into an existing shell. Whereas existing polyethylene liners have not been designed to be re-cemented, this invention includes various features, particularly on the posterior aspect of the liner, to resist pull-out, minimize rotational torsion, and to ensure proper cement mantle thickness.

In the preferred embodiment, the polyethylene liner is disposed within a shell such that the cement locks into features provided on the posterior aspect of the liner which remains in contact with cement. Liners according to the invention further preferably include an extended rim to improve cement pressurization, as well as features to resist torsion as well as pullout, and spacers to ensure proper cement mantle thickness.

### BRIEF DESCRIPTION OF THE DRAWINGS

FIG. 1 is a side-view drawing of an acetabular liner constructed in accordance with the invention; and

FIG. 2 is a cross section of an embodiment of the invention.

### DETAILED DESCRIPTION OF THE INVENTION

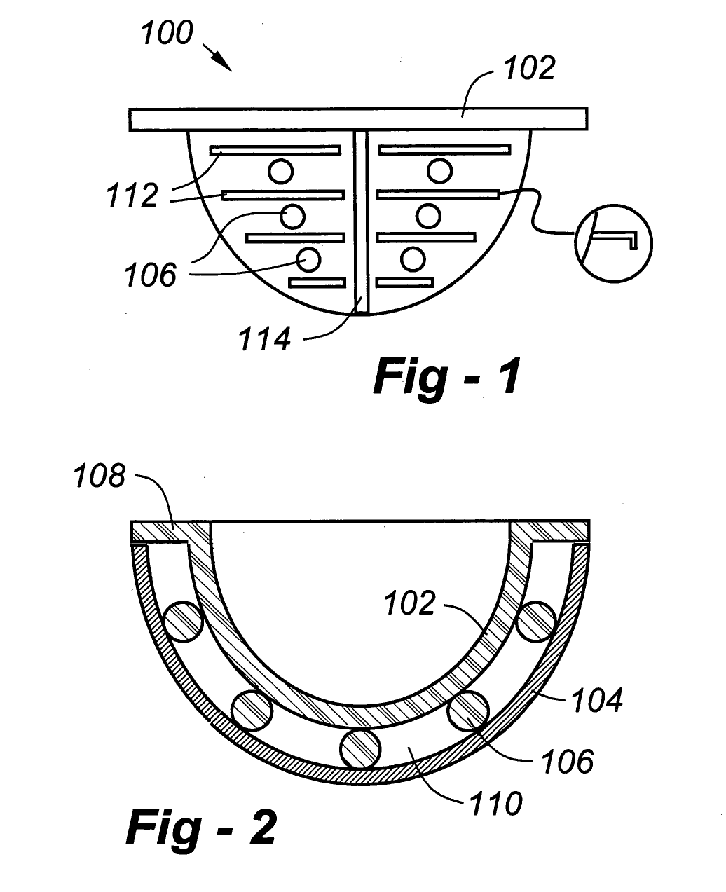

FIG. 1 is a side-view drawing of an acetabular liner constructed in accordance with this invention. The device, shown generally at 100, preferably includes an extended rim 102 to contain cement pressurization, much like existing Ogee liners. However, in conjunction with this feature, the liner includes spacers 106 to prevent bottoming out of the liner within the cup shell, and to ensure an optimum cement mantle, typically on the order of two to four millimeters, or thereabouts.

The preferred embodiment further includes a plurality of raised portions 112, preferably with undercuts to resist pull-out. Additionally, a vertical raised portion 114 may be provided to resist rotation for improved torsional stability.

FIG. 2 is a drawing of an ace tabular liner constructed in accordance with this invention, showing the way in which the liner is contained within a shell 104, and wherein spacers 106 provide a cement mantle 110 of appropriate thickness.

1. An acetabular liner for revision hip surgery, comprising a cup-shaped body having a posterior surface; and
one or more features on the posterior surface to resist pull out, resist torsion, or ensure a desired cement mantle thickness.

2. The acetabular liner of claim 1, wherein the feature to resist pull out includes raised rib portions, each with an undercut.

3. The acetabular liner of claim 1, wherein the features include a vertical oriented rib to resist rotational torsion.

4. The acetabular liner of claim 1, wherein the features include bumps or projections of a predetermined height to set cement mantle thickness.

5. The acetabular liner of claim 1, further including an enlarged rim to enhance cement pressurization.
